(19) United States
(12) Patent Application Publication
(10) Pub. No.: US 2010/0211168 A1
(43) Pub. Date: Aug. 19, 2010

(54) FLEXIBLE VISUAL PROSTHESIS AND METHOD FOR MANUFACTURING A FLEXIBLE VISUAL PROSTHESIS

(76) Inventors: Michael Goertz, Geldern (DE); Hoc Khiem Trieu, Kamp-Lintfort (DE); Wilfried Mokwa, Krefeld (DE)

Correspondence Address: SCHOPPE, ZIMMERMANN , STOCKELER & ZINKLER;C/O KEATING & BENNETT, LLP, 1800 Alexander Bell Drive, SUITE 200, Reston, VA, 20191, US

(21) Appl. No.: 12/676,623

(22) PCT Filed: Aug. 27, 2008
(86) PCT No.: PCT/EP08/07017

§ 371 Date: Mar. 5, 2010

(30) Foreign Application Priority Data
Sep. 7, 2007 (EP) 07017617.7

(51) Int. Cl. A61F 2/16

(52) U.S. Cl. 623/6.22

(57) ABSTRACT

A visual prosthesis for an eye having a lens and a retina has first, second and third substrate parts. The first substrate part has a coil and is fixable in the lens. The second substrate part has stimulation electrodes and connection lines and is fixable to the retina. The third substrate part has a processing unit and connects the first and second substrate parts to each other, the processing unit being electrically connected to the coil and to the stimulation electrodes and being configured to convert a signal generated in the coil by an electromagnetic wave to a stimulation impulse and pass same on via the connection line to the stimulation electrodes. The first substrate part is foldable relative to the third substrate part.

### BACKGROUND OF THE INVENTION

The present invention relates to a flexible visual prosthesis and a method for manufacturing a flexible visual prosthesis and, in particular, to an epiretinal visual prosthesis enabling patients gone blind to visual perception.

An epiretinally implantable visual prosthesis is based on an electrical stimulation of gangliar cells in patients who have, for example, gone blind due to a degeneration of upstream retinal neurons. Situations of this kind occur in Retinitis Pigmentosa (RP), for example, but also in advanced cases of macula degeneration. However, even in advanced cases of Retinitis Pigmentosa and despite a clinically frequently detectable atrophy of the optic nerves, there is frequently still a sufficient number of gangliar cells which can be utilized or recruited for functional electro-stimulation.

The concept of epiretinal visual prostheses (implants implanted in the eye) consists in electrical stimulation of gangliar cells, namely from the ocular inside of the retina. What is necessitated here is a matrix or an array of as many stimulation electrodes as possible of the smallest size possible which are applied onto the retinal surface. Stimulation pulse patterns (electrical stimuli) corresponding to visual impression are released into the nerve tissue at the stimulus electrodes, which result in the most optimal activation possible of the gangliar cells.

Capturing the image data may, for example, take place by means of a camera (by a CMOS camera chip), wherein the camera may, for example, be integrated into an eyeglass. Those stimulation impulses suitable for activating the gangliar cells such that an image as close to reality as possible may form are detected from these images by a visual processor (processing unit). The stimulation signal is encoded, modulated and transmitted to the implant by means of an external transmitter, which may, for example, also be integrated into the glasses. After retrieving the data in receive electronics (receive part+electronics) arranged in the implant (visual prosthesis), electrode-specific bi-phase impulses electrically innervating the gangliar cells via a bio-compatible electrode array are generated by the stimulation electronics.

The implant inside the eye comprises several constituents which may exemplarily comprise a receive part in an artificial lens and a stimulator on the retina. Energy and data for the implant are transmitted wirelessly electromagnetically (exemplarily by means of radio waves) from the transmitter outside the eye to the receive part and, from there, passed on to the stimulator via a micro cable connection after retrieving the data. The stimulator may exemplarily comprise the stimulation electrodes and stimulation electronics converting the information coming from the receive electronics or received by the receive electronics.

In visual prostheses available at present, the electronics components, a receive chip, a stimulator chip, SMD (surface mounted device) elements and a receive coil wound by hand have been mounted onto a thin flexible plastic film and, subsequently, encapsulated using silicone. The receive part here is implemented as an artificial lens and the stimulator chip is arranged close to the stimulation electrodes. In order to make the system bio-compatible, the components have been surrounded by a cover body made of silicone which only exposes the stimulation electrodes.

For introducing such a conventional system into an eye, at first, the lens of the eye is removed via an opening in the cornea (comparable to cataract surgery/operation). Subsequently, the vitreous body is removed and the capsular bag is opened for introducing the stimulation part. The stimulator part is then pushed into the inside of the eye via the opening in the cornea and the opening in the capsular bag. After that, the artificial lens is placed in the capsular bag. Very large openings are necessitated due to the size of the implanted electronical components, a fact that increases surgical risks significantly.

In a further development, it was possible to miniaturize these conventional systems. The coil of this further-developed system is no longer wound by hand, but applied onto a flexible carrier using microgalvanics. Additionally, the chips are outside the lens and it is therefore possible to fold the artificial lens. Flip-chip technology is employed for mounting the chips and, by using the microcoil, it has become possible to fold the region of the artificial lens. This has resulted in a considerable reduction in the opening for introducing the artificial lens.

However, the further-developed systems are of disadvantage in that both the SMD elements and the two chips are arranged on the cable part which connects the coil to the stimulation electrodes. Due to the stiffness of these elements, however, the flexibility is decreased significantly in the area in which the elements and chips are arranged. This makes implanting these further-developed systems considerably more difficult. Due to the structural height of the individual components, a relatively large opening in the capsular bag will still be necessitated for introducing the implant into the eye.

Additionally, the stimulation electrodes are very difficult to place precisely due to the weight of the elements (SMD elements, chips) and consequently the film may be fixed additionally in the area of the electronical elements.

### SUMMARY

According to an embodiment, a visual prosthesis for an eye having a lens and a retina may have: a first substrate part having a coil and being fixable in the lens; a second substrate part having stimulation electrodes and connection lines and being fixable to the retina; and a third substrate part having a processing unit and connecting the first and second substrate parts, the processing unit being electrically connected to the coil and to the stimulation electrodes and being configured to convert a signal generated in the coil by an electromagnetic wave to a stimulation impulse and pass same on via the connection line to the stimulation electrodes, wherein the first substrate part is foldable relative to the third substrate part and the third substrate part has a ring-shaped or crescent-shaped form so that a central area remains open after folding the first substrate part relative to the third substrate part.

According to another embodiment, a visual prosthesis for an eye having a lens and a retina may have: a first substrate part having a coil and being fixable in the lens; a second substrate part having stimulation electrodes and connection lines and being fixable to the retina; and a third substrate part having a processing unit and connecting the first and second substrate parts, the processing unit being electrically connected to the coil and the stimulation electrodes and being configured to convert a signal generated in the coil by an electromagnetic wave to stimulation impulses and pass same on to the connection line, wherein the second substrate part has a mounting aid which is configured to engage a further adjusting aid connected to the retina.

According to another embodiment, a visual prosthesis for an eye having a lens and a retina may have: a first substrate part having a coil and being fixable in the lens; a second substrate part having stimulation electrodes and connection lines and being fixable to the retina; and a third substrate part having a processing unit and connecting the first and second substrate parts, the processing unit being electrically connected to the coil and to the stimulation electrodes and being configured to convert a signal generated in the coil by an electromagnetic wave to a stimulation impulse and pass same on via the connection line to the stimulation electrodes, wherein the first substrate part is foldable relative to the third substrate part, and wherein the second substrate part has a mounting aid having a positioning aid, the mounting aid being configured to engage a further adjusting aid connected to the retina, and the positioning aid being configured to allow relative positioning of a stimulator relative to the further adjusting aid by the positioning aid having an inner diameter which is as great as or greater than the adjusting aid.

According to another embodiment, a method for using a visual prosthesis as mentioned above may have the steps of: detecting an image by a camera; generating a pattern of the detected image; transmitting the pattern by transmitting means to the visual prosthesis; generating a stimulation impulse by the processing unit; and passing the stimulation impulses on to the stimulation electrodes.

According to another embodiment, a method for manufacturing a visual prosthesis for an eye having a lens and a retina may have the steps of: providing a first substrate part, the first substrate part having a coil and being fixable in the lens; providing a second substrate part having stimulation electrodes and connection lines and being fixable to the retina; and connecting the first substrate part and the second substrate part by a third substrate part, the third substrate part having a processing unit so that the coil is connected to the stimulation electrodes, so that a signal generated in the coil by an electromagnetic wave is converted to a stimulation impulse and passed on to the connection lines, wherein the third substrate part may be foldably connected to the first substrate part.

According to another embodiment, a method for attaching a visual prosthesis having first, second and third substrate parts at a predetermined position in an eye having a lens and a retina may have the steps of: folding the first substrate part relative to the third substrate part; potting the folded first and third substrate parts using silicone; folding the potted first and third substrate parts along a line which is parallel to a direction of introducing the visual prosthesis into the eye; introducing the visual prosthesis into the eye; fixing the folded first and third substrate parts in the lens; and fixing a second substrate part having stimulation electrodes and a connection line to the retina.

The present invention is based on the finding that a visual prosthesis for an eye having a lens and a retina comprises a substrate having first, second and third substrate parts, wherein the first substrate part comprises a coil and is fixable in the lens, the second substrate part comprises stimulation electrodes and a connection line and is fixable to the retina, the third substrate part comprises a processing unit and connects the first and second substrate parts such that the first substrate part is foldable relative to the third substrate part. In addition, the processing unit is connected electrically to the coil and to the stimulation electrodes and configured to convert a signal generated in the coil by an electromagnetic wave to stimulation impulses and pass on the stimulation impulses to the stimulation electrodes.

In embodiments of the present invention, folding may be performed such that the third substrate part which comprises the processing unit is fixable in the lens together with the first substrate part and the connection lines make an electrical connection to the second substrate part and, thus, to the stimulation electrodes. Here, the first substrate part may comprise a flexible film in the shape of a circular disk or a circular ring having outer and inner circle edges, the coil being wound along the circular disk. Thus, exemplarily, the first substrate part when folded onto the third substrate part can be folded again so that only a small slit will be necessitated in the eye (or the lens) when implanting. The second folding may, for example, take place in parallel to an implantation direction or in parallel to the connection line. Thus, the visual prosthesis can be folded twice before being implanted, which makes implantation considerably easier.

Further embodiments comprise detachable electrode mountings and positioning aids which support easy fixing and detaching of the stimulator. Exemplarily, the electrode mountings may comprise lateral openings so that detaching the stimulator from the retinal nails or pins may be performed by simply pushing the stimulator out (without pulling the retinal nails). Conversely, easy mounting may also take place by simply pushing in. Additionally, the electrode mountings may also comprise a longitudinal hole so that the stimulator may also be mounted to a retinal nail by pushing up or down.

The serious disadvantages mentioned above are removed or overcome by embodiments of the present invention. In particular, it becomes possible to shift all the electronical components in the area of the artificial lens (or lens), so that flexibility of the film can be maintained from the lens to the stimulation electrodes. This is, in particular, possible since only the connection part is arranged between the components arranged inside the lens and the stimulation electrodes mounted to the retina and since this connection part may comprise a flexible film (including embedded conductive traces).

The operatively necessitated opening can be minimized considerably by this—in particular when the film is exemplarily extended in a crescent-shaped manner in the area of the artificial lens (or devices) or comprises a crescent-shaped part and is provided with conductive traces for electrically contacting the electronical devices. After contacting and fixing the devices, the coil can be folded by 180 degrees onto the crescent-shaped part including the electronical elements. The crescent-shaped section thus should be implemented such that the round shape of the film remains after folding in the area of the artificial lens. The clearance between the coil and the components may be filled with a suitable glue so that, after folding, the film with the coil (first substrate part) and the crescent-shaped part (third substrate part) are glued onto each other. Folding may be performed such that, after folding, the inside within the inner circle edge of the first substrate part is left empty. This can be achieved by means of a suitable implementation of the crescent-shaped part. After folding and optical gluing, the folded first and third substrate parts may also be potted using silicone (or another material), the result being encapsulation.

Embodiments of the present invention thus include a number of advantages compared to conventional implants described before. Caused by the crescent-shaped arrangement, on the one hand, the central opening of the first substrate part remains empty so that visual observation of the retina is possible. Furthermore, due to the crescent-shaped form of the third substrate part, when folded, one half of the first substrate part shaped as a circular disk remains foldable, i.e. the lens remains half-foldable by the semicircular arrangement achieved in this way. Additionally, it is possible, when more space for electronical components is necessitated, to add further crescents which may also be folded onto one another so that further substrate parts can be folded one above the other. Another advantage of this arrangement is that all the components including the coil can be protected on both sides by the flexible film and be potted using a glue. Thus, the effectivity of the encapsulation, for example, can be increased significantly with regard to both an effective mechanical protection and with regard to humidity penetrating.

It is also of advantage that the folding edge may, for example, be predetermined or made easier by providing neckings in the film in the area of transition in the coil area (first substrate part) and the area of transition to the electrode lines (connection lines). Additionally, it is possible to achieve precise and reproducible folding by applying corresponding adjusting marks in the coil part and also in the crescent-shaped part (second substrate part). Additionally, any angle between the artificial lens and the outgoing lines can be set by bending the connection part between the artificial lens and the stimulation electrode array (stimulation electrodes). This is possible because the connection part is shaped to be flexible. In addition, this makes placing the electrode array on the retina easier.

Finally, the inventive implementation of the electrode mounting including positioning aids is of advantage in that the retina nails or pins do not have to be removed with potentially explanting of the visual prosthesis and in that simply pushing the stimulator in and out becomes possible. The combination of the electrode mounting and the positioning aids is additionally of advantage in that the position of the stimulator below the retina nails can be determined precisely.

Other elements, features, steps, characteristics and advantages of the present invention will become more apparent from the following detailed description of the preferred embodiments with reference to the attached drawings.

### BRIEF DESCRIPTION OF THE DRAWINGS

Embodiments of the present invention will be detailed subsequently referring to the appended drawings, in which:

FIG. 1 shows a visual prosthesis according to an embodiment of the present invention;

FIG. 2 shows a visual prosthesis after folding;

FIG. 3 shows a systematic concept of the epiretinal visual prosthesis;

FIGS. 4A, B show a top view and a cross sectional view, respectively, of folding supporting means;

FIG. 5 shows a mechanical adjusting aid in accordance with an embodiment of the present invention;

FIG. 6 shows adjusting a retinal nail using the positioning aid;

FIG. 7 shows a cross sectional view of a three-dimensional electrode structure.

### DETAILED DESCRIPTION OF THE INVENTION

With regard to the following description, it should be kept in mind that same functional elements or functional elements having the same effect are given the same reference numerals in different embodiments and that a repeated description of these elements is omitted.

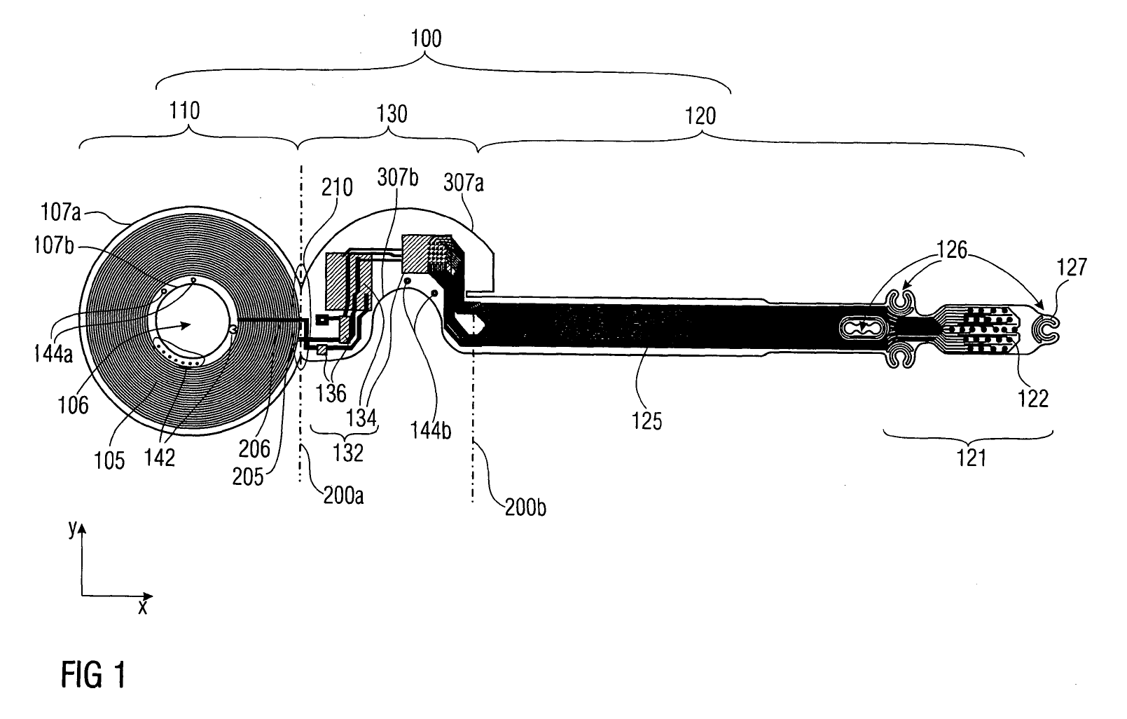

FIG. 1 shows a visual prosthesis 100 or implant in accordance with an embodiment of the present invention. The visual prosthesis 100 comprises a first substrate part 110 and a second substrate part 120 which are connected to each other by a third substrate part 130.

The first substrate part 110 comprises a coil 105 which has a disk-like circular ring structure and leaves a central area 106 empty. Lines of the coil 105 contacted by a first and a second connection line 205, 206 are thus wound circularly between an outer circle edge 107a and an inner circle edge 107b leaving the central area 106 open.

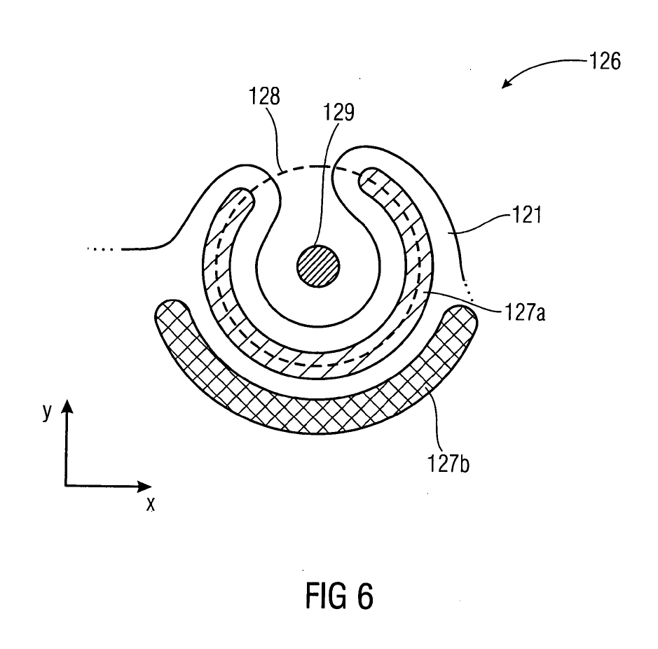

The second substrate part 120 comprises a stimulator 121 including stimulation electrodes 122 which are contacted by connection lines 125. The stimulator 121 including the stimulation electrodes 122 serves for stimulating the retina or, more precisely, the gangliar cells and can be mounted to the retina using mounting aids 126 (electrode mounting). For improved orientation and adjusting, the electrode mountings 126 comprise positioning aids 127 (see bottom of FIG. 6). The third substrate part 130 comprises a processing unit 132 which may, for example, comprise SMD components 136 and semiconductor circuits 134.

The third substrate part 130 may be implemented to be crescent-shaped so that the third substrate part 130 comprises two semicircular edges—an outer crescent circle 307a and an inner crescent circle 307b. The processing unit 132 is arranged along the crescent and generates stimulation signals for the individual stimulation electrodes 122. The third substrate part 130 is connected to the first substrate part 110 along a first separation line 200a and to the second substrate part 120 along a second separation line (or further separation line) 200b, wherein in FIG. 1 the first to third substrate parts 110, 120, 130 are arranged in an (x, y) plane along the x direction such that the first and second separation lines 200a and 200b are in parallel to the y axis. However, in further embodiments, the separation lines 200 can be oriented differently. Folding supporting means 210 is formed along the first separation line 200a to support folding along the first separation line 200a.

For improved adjusting of the first and third substrate parts 110, 130 after folding, optionally, a first adjusting aid (adjusting mark) 144a which may comprise several components may be formed at the first substrate part 110 and a second adjusting aid 144b which may also comprise several components may be formed at the third substrate part 130. The adjusting aid 144 is formed such that, after folding, the first adjusting aid 144a and the second adjusting aid 144b can be matched and similarly, when present, further components can also be matched. The first adjusting aid 144a and implant labeling and encoding 142, for example, too, may be arranged along the inner circle edge 107b.

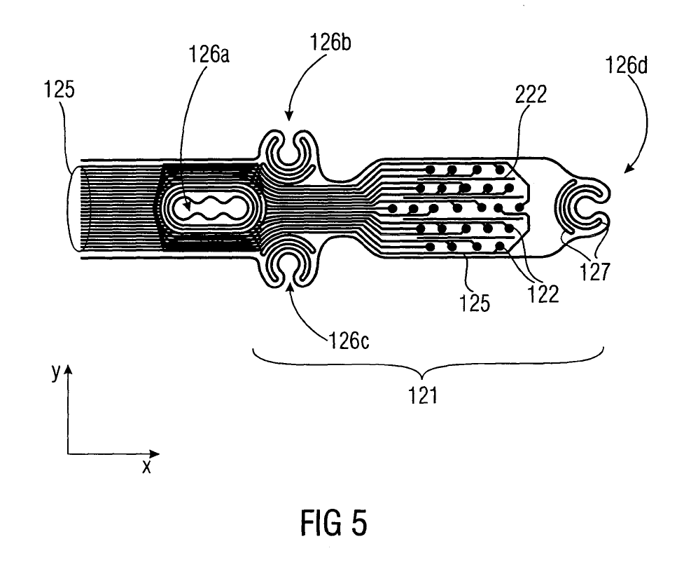

The connection between the third substrate part 130 and the second substrate part 120 along the second separation line 200b is formed such that the second substrate part 120 and the third substrate part 130 are foldable along the second separation line 200b. The connection lines 125 realizing contacting of the stimulation electrodes 122 (connection between the processing unit 132 and the stimulation electrodes 122) may, for example, be strengthened galvanically so that an electrical resistance can be kept as small as possible. Furthermore, the electrode mounting 126 can be implemented in a detachable manner, which will be described below in greater detail in FIG. 5.

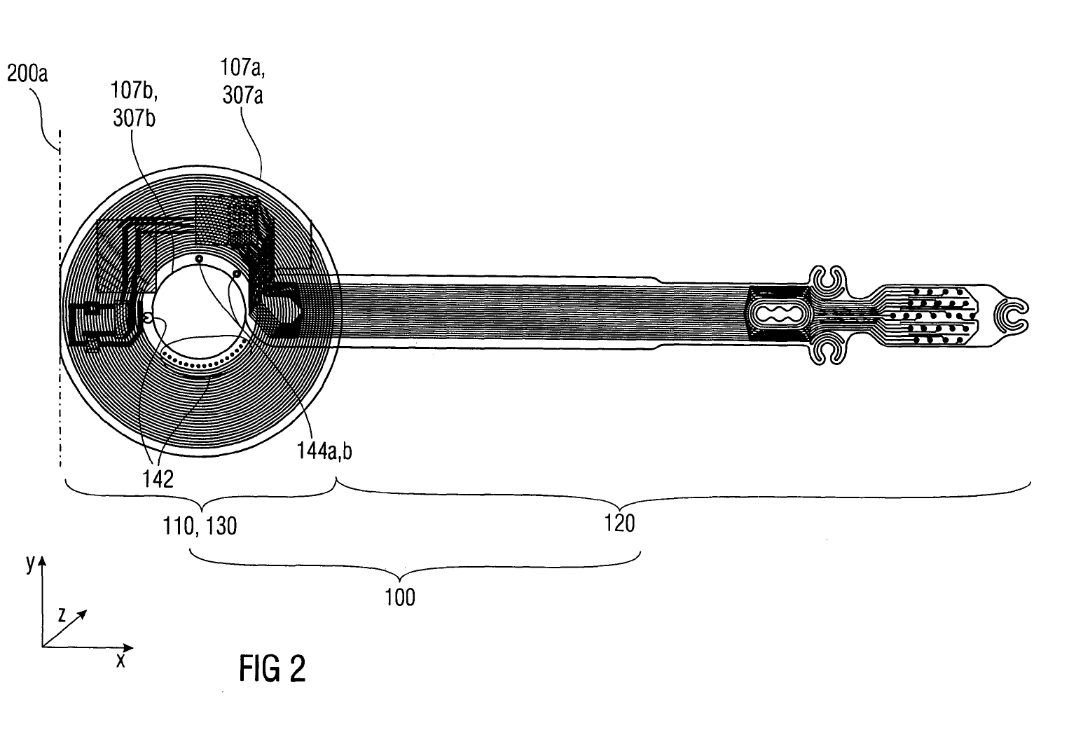

FIG. 2 shows the result of folding the first substrate part 110 relative to the third substrate part 130 along the first separation line 200a. Since the third substrate part 130 may comprise, as shown in FIG. 1, a crescent-shaped form, the outer crescent circle 307a can be shaped such that, after folding along the first separation line 200a, the outer crescent circle 307a can be matched with the outer circle edge 107a. Similarly, the inner crescent circle 307b may be shaped such that, after folding along the first separation line 200a, the inner crescent circle 307b can be matched with the inner circle edge 107b.

Thus, as a consequence of the crescent-shaped character of the third substrate part 130, when folding along the first separation line 200a, the third substrate line 130 can be covered by the first substrate part 110 such that, in particular, the central area 106 remains free.

For correctly adjusting the first and third substrate parts 110, 130 when folding, the adjusting aids 144a and 144b can be used. Exemplarily, the first adjusting aid 114a may be implemented as an edge of a geometrical figure (such as, for example, a circle, quadrangle, etc.) and the second adjusting aid 114b may, for example, be implemented as the geometrical figure. Thus, it is possible easily to match, for example, a circle (first adjusting aid 144a) with a circular disk (second adjusting aid 144b). The circle may comprise a greater radius than the circular disk (the circular disk may, for example, also be a point), which makes centering the circular disk within the circle easier. The same principle may also be applied to other geometrical figures.

Additionally, it is of advantage for the third substrate part 130 to be transparent along the inner crescent circle 307b so that the implant labeling and encoding 142 are visible after folding the third substrate part 130 relative to the first substrate part 110. Another way is arranging the implant labeling and encoding 142 in an edge area along the inner circular edge 107b which is still exposed after folding the third substrate part 130, like for example in that part along the inner circular edge 107b which is not matched with the inner crescent circle 307b when folding along the first separation line 200a. As is shown in FIG. 2, implant labeling and encoding 142 are still clearly detectable.

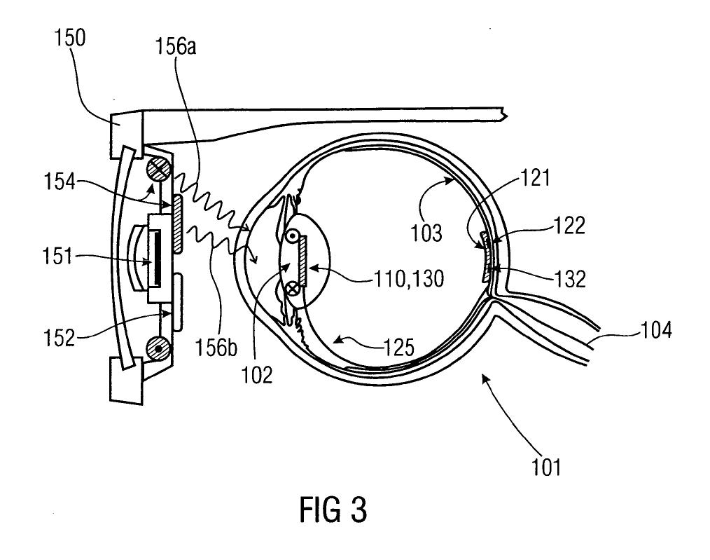

FIG. 3 shows a systematic concept for an epiretinal visual prosthesis 100 as has exemplarily been described in FIG. 1. What is shown is an eye 101 comprising a lens 102, a retina 103 and an optic nerve 104. The lens may also be implemented as an artificial lens. The visual prosthesis 100 is transplanted into the eye such that the first and third substrate parts 110 and 130 are fixed in the lens 102 and the connection lines 125 make contact to the stimulator 121, wherein the stimulator 121 (or retina stimulator) may exemplarily be mounted to the retina in that area where an image is generated in a healthy eye. In order to be able to generate an image by stimulating the retina, an image may be generated at first (like for example by a stimulation pattern encoder), which may, for example, be performed by means of special glasses 150. The glasses 150 here exemplarily comprise a camera 151 which may exemplarily include a single-chip CMOS camera. Additionally, the glasses comprise a signal processor 152 which exemplarily processes (like for example encodes and modulates) the electronical image data generated by the camera 151 correspondingly. The data achieved in this way can then be transmitted from a data transmitter 154 (telemetry transmitter) to the visual prosthesis 100. Transmission may, for example, take place by means of an electromagnetic wave 156a, but also optically 156b. The frequency of the electromagnetic wave 156a for transmitting data may, for example, be in a range between 0.1 and 100 MHz or between 10 and 15 MHz or be around 13.56 MHz. The electromagnetic wave 156a is received by the coil 105 in the first substrate part 110 and converted to an electrical impulse which is then evaluated by the processing unit 132.

The electromagnetic wave 156a may, apart from the image data generated by the camera 151, also transmit the energy for operating the visual prosthesis 100. This may, for example, take place similarly to what is conventionally done in RFID (radio frequency identification) systems. The coil 105 in the first substrate part 110, as telemetry receiver, serves both for receiving data and for supplying energy (receiving supply energy) and is, for example, fixable in the lens 102. The data received are then transferred to the stimulator 121 via the connection lines 125 which may, for example, comprise microcables, wherein the stimulator 121 may comprise an array of stimulation electrodes 122 and, optionally, stimulator electronics. The stimulator 121 may comprise a corresponding encapsulation which exemplarily offers protection from liquid present along the retina. After stimulating the retina 103 by the electrical impulses applied to the stimulator 121 and generated in correspondence with the image data as detected by the camera 151, the optic nerve 104 can pass corresponding impulses on to the brain and thus trigger visual perception.

Thus, apart from the visual prosthesis 100, embodiments of the present invention include a stimulation pattern generator which may exemplarily be arranged in the glasses 150 and comprises telemetry for data transmission and energy supply. Data transmission may also take place optically. In this case, the visual prosthesis 100 also necessitates means for receiving optical data, like for example by means of an optical sensor implemented to receive and process the optical signals emitted by the transmitting means 154. Optical signal transmission may, for example, take place by means of a special laser (like for example in the infrared range).

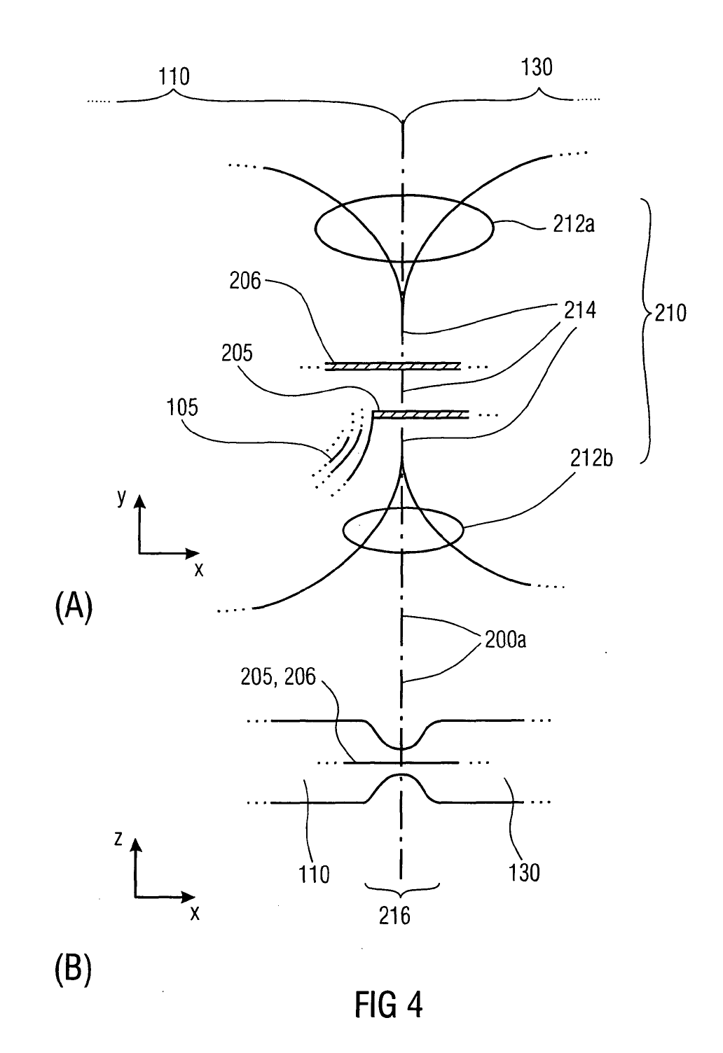

FIGS. 4A, B show an embodiment of folding supporting means 210 which may exemplarily be formed along the first separation line 200a. The first separation line 200a separates the first substrate part 110 from the third substrate part 130 (on the right side of FIG. 4).

FIG. 4A shows a top view of that portion of the visual prosthesis 100 arranged in the (x, y) plane. This image portion also shows a section of the coil 105 which is contacted by the first connection line 205, wherein the second connection line 206 contacts the coil 105 from the other end (not shown in FIG. 4) (see FIG. 1). The folding supporting means 210 comprises, on the one hand, first and second notches 212a, 212b in the transition region of the substrate from the first substrate part 110 to the third substrate part 130. Additionally, the folding supporting means 210 may comprise perforations 214 so that the substrate comprises holes or trenches so as to make folding along the first separation line 200a easier.

FIG. 4B shows the same image portion, but shifted to the (x, z) plane so that a cross section through the visual prosthesis 100 is shown to be in parallel to the x-axis. Again, the first separation line 200a separates the first substrate part 110 from the third substrate part 130. The folding supporting means 210 comprises a necking 216 in the transition region between the first substrate part 110 and the third substrate part 130 so that the substrate comprises a smaller layer thickness close to the necking 216 and thus folding along the first separation line 200a is made easier.

In further embodiments, the folding supporting means 210 is shaped differently or only part of the means shown in FIG. 4, namely the notches 212a, 212b, the perforations 214 and the necking 216, is realized. The folding supporting means 210 is implemented such that the first and second connection lines 205, 206 or further connection lines not shown in FIG. 4 are not damaged by the folding supporting means 210, or interrupted. Exemplarily, the first and the second connection lines 205, 206 are implemented such that, after folding, a perfect current flow or perfect signal transmission can be ensured.

FIG. 5 shows the retina stimulator 121 which is contacted by the connection lines 125. The retina stimulator 121 exemplarily comprises the stimulation electrodes 122 which may exemplarily be contacted one by one by a respective one of the connection lines 125 and arranged in the stimulator 121 as an array. In the embodiment shown in FIG. 5, the array of the stimulation electrodes 122 is implemented such that, in five rows, a first row comprises four electrodes, a second row comprises five electrodes, a third row comprises 7 electrodes, a fourth row comprises five electrodes, and a fifth row comprises four electrodes. The individual rows of the array may optionally be separated from one another by separation walls 222. The separation walls 222 may exemplarily have a shielding effect so that electrical stimulations of a first region are limited and further electrical stimulation of a second region is suppressed by the separation walls 222. Thus, improved insulation/isolation of the retinal regions electrically stimulated by the stimulation electrodes 122 can be achieved for example.

In FIG. 5, exemplarily only one shape is shown for the array and in further embodiments, the array may also comprise a different shape (exemplarily arranged to be quadrangular or rectangular or even circular) or the number of stimulation electrodes 122 may also be varied. Furthermore, in further embodiments, the mean lateral distance of the stimulation electrodes 122 along the x direction may be variable.

The electrode mounting 126 which may have different shapes serves for fixing the stimulator 121. Exemplarily, a first electrode mounting 126a may be implemented to be longitudinal or slit-shaped as a longitudinal hole so that shifting the stimulator 121 in parallel to the x-axis is still possible—at least within the limits set by the longitudinal extension. A second electrode mounting 126b may exemplarily be shaped such that pushing the stimulator 121 in below a pin or nail can be ensured along the positive y-axis. A third electrode mounting 126c is implemented in a similar manner, however with the difference that shifting the stimulator 121 down below a pin or nail is allowed in the negative y direction. A fourth electrode mounting 126d in turn is implemented to be semicircular comprising an opening along the positive x-axis so that pushing the stimulator 121 in or down below a nail or pin is allowed along the positive x-axis. Thus, the second, third and fourth electrode mountings 126b, 126c, 126d are all implemented to be circular, however with openings along different directions. Pushing the stimulator 121 out or in without removing the pins or nails is possible here. This is, above all, supported by the fact that the stimulator 121 and the connection line 125 may comprise a flexible substrate (as a film).

FIG. 6 shows an electrode mounting 126 which comprises a positioning aid 127, wherein the positioning aid 126 serves for positioning the stimulator 121 relative to a retina nail 129 having a nail head 128. The electrode mounting 126 in this embodiment is implemented to be circular, wherein the electrode mounting 126 comprises an opening along the positive y-axis so that pushing in and out below a nail head 128 becomes possible along the positive and negative y-axes. The optimum position of the stimulator 121 may exemplarily be determined by the nail head 128 or the boundary curve thereof (broken line in FIG. 6) to be as concentric in the positioning aid 127 as possible. This may exemplarily correspond to that case in which the nail or pin 129 is as centralized as possible within the circularly implemented electrode mounting 126.

The positioning aid 127 may additionally comprise several parts, wherein a first part 127a may be used for a first position of the nail 129 and the second part 127b may be used for a second position of the nail 129. In both cases, the first and second positions may for example by reached when the nail head 128 or the outer boundary line thereof is concentric or at a fixed predetermined position relative to the first and second parts 127a and 127b of the positioning aid 127. The parts of the positioning aids 127 may, of course, also be used for fixing the stimulator 121 at other positions, for example when the boundary line is not concentric but located at another certain position.

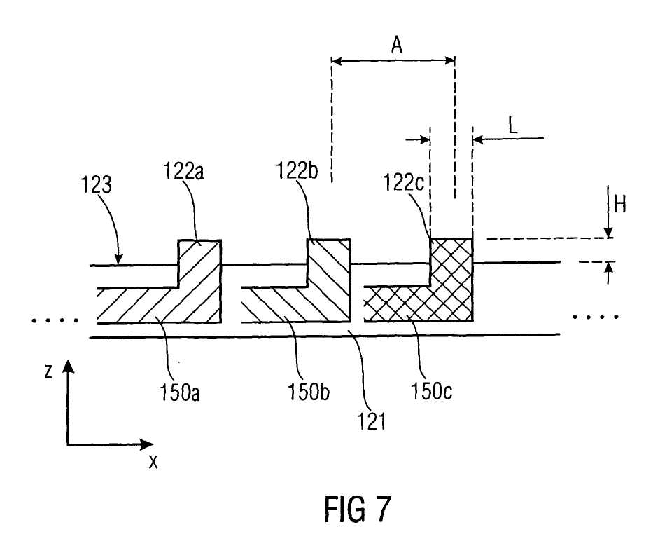

FIG. 7 shows an embodiment of a three-dimensional implementation of the stimulation electrodes 122 along the stimulator 121. FIG. 7 shows a cross sectional view in the (x, z) plane, wherein part of the stimulator 121 comprising a first stimulation electrode 122a, a second stimulation electrode 122b and a third stimulation electrode 122c is shown. The first to third stimulation electrodes 122a to 122c are arranged along the x direction, wherein the first stimulation electrode 122a is contacted by a first connection line 150a, the second stimulation electrode 122b is contacted by a second connection line 150b and the third stimulation electrode 122c is contacted by a third connection line 150c. The first, second and third connection lines 150a, 150b, 150c are not in direct electrical contact and are correspondingly passed on in an offset manner next to one another along the y direction (perpendicular to the direction of the drawing).

As the embodiment in FIG. 7 shows, the stimulation electrodes 122 can be implemented to be three-dimensional, wherein the three-dimensional implementation refers to the fact that the stimulation electrodes 122 do not terminate on the surface 123 of the stimulator 121, but extend beyond up to a height H above the surface 123. In addition, the stimulation electrodes 122 exemplarily comprise a mean lateral extension L along the x direction and another lateral extension along the y direction which, however, is not shown in FIG. 7. The distance A of neighboring stimulation electrodes 122 may vary and be adjusted correspondingly.

In the three-dimensional implementation of the stimulation electrodes 122, the height H may, for example, be at least 10 or at least 50% of the lateral extension L or the further lateral extension along the y direction. It is also possible for the height H to be selected to be greater than the lateral extension L or selected to be greater than the further lateral extension or selected to be greater than the maximum of the lateral extension L and the further lateral extension. The three-dimensionally implemented stimulation electrodes 122 may exemplarily also comprise gold and the height H may exemplarily be greater than 10 μm or at least 30 μm or be in a range between 30 μm and 40 μm.

Thus, in embodiments of the present invention important functionalities which are of great importance for a future usage of the implant (visual prosthesis 100) may be implemented in a film (substrate) comprising a first substrate part 110, second substrate part 120 and third substrate part 130. This exemplarily includes the coil part (first substrate part 110) and the crescent-shaped part of the film (third substrate part 130) which may exemplarily contain metallic structures for individually encoding the implants. By correspondingly removing parts of these metallic structures during assembly of the elements, each implant can be provided with a unique number. The encoding structures may exemplarily be placed such that they are still detectable after encapsulation and even after implantation.

Digital encoding for identifying the implant may exemplarily take place such that metallic points (for example five) are provided on the first substrate part and encoding can be done by removing individual ones of or a group of or all points. With n metallic points, this would allow 2ⁿ encodings (with n=5, for example, there is a total of 32 encodings). Additionally, a number may be provided for identification (like for example a serial number). Encoding by means of removing points is of advantage in that this can be done by a surgeon when implanting and allows future conclusions as to the point in time and location of and as to who has implanted the implant.

Another functionality results from the fact that the stimulator part 121 is fixed on a retina 103 during surgery via so-called retinal nails 129. Openings (positioning aids 126) for these nails 129 are provided close to the stimulation electrodes 122 or the stimulation electrode array. In order to allow future explantation of the implant (visual prosthesis 100), longitudinal holes 126 are provided in the central region of the film and holes having a lateral opening 126b, 126c, 126d are provided in the boundary area of the film. The holes have a diameter in the size of the retina nail pins 129. The film is fixed on the retina 103 by the nail head 128. Due to the longitudinal geometry of the electrode mounting 126a or the lateral opening of the holes (electrode fixings 126b, 126c and 126d), when explanting, the film can be peeled off over the nail head 128. The nail 129 thus may remain in the retina 103. Traumatic damages of the retina caused by pulling out the nail or nails 129 can be avoided by this. The number of nails 129 (one or several nails) may vary in different embodiments.

Another functionality is given by the fact that the nail head 128 is considerably greater compared to the diameter of the opening. This causes the opening to be covered by the nail head 128 when nailing (see FIG. 6), before the nail or nail pin 129 reaches the opening so that the surgeon can no longer see the position of the nail pin 129 relative to the opening. Nailing then takes place in a “blind” manner. In order to be able to offer the surgeon an orientation aid, a metal strip (positioning aids 127), either longitudinal or round, the internal diameter of which is selected to be as great as or somewhat greater than the nail head 128 may be realized around the area of the opening. This structure remains visible when nailing and may be used during nailing to allow exact positioning of the stimulator 121, for example.

Additionally, it is possible to strengthen metallic conductive traces 125 galvanically in order to considerably decrease the ohmic resistance and thus reduce heat development within the eye to a minimum.

It is also possible, as shown in FIG. 7, to implement the stimulation electrodes 123 to be three-dimensional to obtain, in contrast to planar electrodes, improved electrical contact to the gangliar cells and thus allow improved electrical stimulation of the retina. It is also possible to use a material for increasing the charge transfer capacity or coat the stimulation electrodes 122 with such a material. Exemplarily, the stimulation electrodes 122 may be coated with metals like iridium, iridium oxide or platinum/platinum black.

The flexible carrier film (substrate comprising the first to third substrate parts 110, 120, 130) and the encapsulation material may exemplarily be implemented to be transparent so that optical transmission becomes possible in addition to electromagnetic transmission of supply energy and data. Optical transmission may exemplarily be used for transmitting data at a higher data throughput. An infrared laser of low energy (or low heat development) may exemplarily be used for this. When using optical transmission, the artificial lens exemplarily comprises an optical receiver (photo cell, photo detector etc.).

Thus, embodiments of the present invention include a visual prosthesis 100 which is made up of a flexible substrate including a coil 105, electronical devices 132 and stimulation electrodes 122 such that, by means of folding the coils 105 onto the area 130 containing the electronical components, these components 132 can be placed completely in the area of the artificial lens 102.

In further embodiments, the electronical components 132 are of a semicircular setup (crescent-shaped) so that, after folding, one half of the lens remains flexible. In further embodiments, the flexible visual prosthesis 100 comprises neckings in the film in the area of the transition between the coil area (first substrate part 110) and in the area for transition to the electrode lines 150 such that the folding direction is predetermined and folding is made easier.

In further embodiments, the area of the artificial lens has an opening 106 in the center so that the surgeon is able to see inside the eye through this opening. Additionally, it is possible for the coil part (first substrate part 110) and/or the area of the electronical devices (third substrate part 130) to be provided with structures 142 for individually encoding the implants. The encoding structures here can be placed such that they can be recognized even after encapsulation and also even after implantation. These structures 142 for individually encoding the implants may exemplarily comprise metal so that each implant can be provided with a unique number by correspondingly removing parts of these metallic structures during assembly of the devices.

Additionally, the flexible visual prosthesis 100 may comprise, close to the stimulation electrodes 122 or the stimulation electrode array 121, longitudinal holes 126a or lateral openings 126b, 126c, 126d for receiving a retina nail 129 in the substrate film so that future explantation of the implant will be possible without removing the retina nail 129 or the pin.

In further embodiments, metallic strips 127, longitudinal or round, the inner diameter of which is as great as or greater than the retina nail head 128 are realized in the area of the openings 126 for receiving the retina nail 129 so that these structures remain visible when nailing and can be used as an orientation aid for a surgeon when nailing or mounting.

The metallic conductive traces 125 forming the connection between the electronical devices 132 and the stimulation electrode array 121 can be strengthened galvanically so as to considerably reduce the ohmic resistance.

In addition, it is possible for the stimulation electrodes 122 to be covered with a material, such as, for example, iridium, iridium oxide or platinum/platinum black, to increase the charge carrier transfer capacity. Furthermore, the stimulation electrodes 122 may comprise a three-dimensional shape or may be implemented to be three-dimensional.

Finally, the visual prosthesis 100, as flexible carrier film and as encapsulation material, may be implemented to be transparent so that optical transmission will be possible in addition to electromagnetically transmitting energy and data.

Since an epiretinal implant in this design has a transparent encapsulation, on the one hand, a surgeon is able to get insight into the inside of the eye through this central opening 106 during surgery and, on the other hand, recognize the implant encoding.

Thus, the present invention relates to an epiretinal visual prosthesis which enables patients gone blind to visual perception. The goal of epiretinally implantable visual prostheses is electrically stimulating gangliar cells or other parts of the retina of the patients who have gone blind due to a degeneration of upstream retinal neurons. Situations of this kind occur, for example, in Retinitis Pigmentosa (RB), but also in advanced cases of macula degeneration.

Embodiments of the present invention are of particular advantage in that they allow sensitive treatment of the retina. This is possible because, by folding, all the heavy or non-flexible parts can be placed in the lens and thus the pressure on the retina is minimized. At the same time, the weight transfer to the lens 102 also offers improved mechanical support of the visual prosthesis.

Gluing the stimulator 121 onto the retina is also possible in principle—but generally difficult—which is why usually the stimulator 121 is fixed to the retina by pins or nails 129. In addition to epiretinal stimulation, sub-retinal stimulation where the stimulator is fixed between different skins/layers of the retina is also possible.

The substrate (film) may exemplarily comprise polyimide and consist of several layers (multi-layer film), wherein the coil 105 may exemplarily be realized within an inner layer. 25 stimulation electrodes are arranged in the stimulator 121 in embodiments, in other embodiments, up to 100 or up to 1,000 or even more stimulation electrodes 122 may be arranged in the stimulator 121. Thus, the stimulation electrodes excite whole areas of the retina.

Folding the first and third substrate parts 110, 130 or further substrate parts may take place such that surfaces of the folded substrate parts are contacted or are connected by a glue layer. It is also possible to fix folding using mechanical fixing (such as, for example, clamping, clicking etc.).

In addition to the embodiments already described, further optional developments are possible. One way is for the visual prosthesis 100 to comprise a further substrate part. The further substrate part may exemplarily comprise a further coil which is connected in series or in parallel to the coil 105 of the first substrate part 110. The further substrate part may exemplarily be arranged such that the first substrate part 110 is arranged between the further and the third substrate parts 130 and thus the further substrate part, together with the first and third substrate parts 110, 130, is foldable onto one another. When connecting the coil 105 and the further coil in series, a coil of an increased number of windings is generated effectively after folding, while, when connecting in parallel, the coil impedance of the effective coil is reduced. In the same way, additional substrate parts may be added, which is how the inductivity of the coil can be increased significantly at the same time.

It may also be of advantage for the visual prosthesis 100, before implanting and gluing, to be used at first outside the eye for test purposes. The respective method may exemplarily comprise the following steps: detecting an image using the camera 151, generating a pattern based on the detected image, transmitting the pattern by the transmitting means 154 to the visual prosthesis 100, generating a stimulation impulse by the processing unit 132 and passing the stimulation impulse on to the stimulation electrodes 122.

Finally, in particular for a greater number of stimulation electrodes 122, another advantageous implementation may be for not each stimulation electrode 122 to be controlled individually via a connection line 125, but for a multiplex line (or a bus) to transmit the signals or patterns for at least a part of stimulation electrodes 122. Here, the second substrate part 120 may comprise a control chip (or demultiplexer) which receives the signals from the multiplex line and converts same to stimulation impulses for the individual stimulation electrodes (which are components of the part of the stimulation electrodes 126). On the other hand, in this embodiment, the processing unit 132 may comprise a multiplexer which exemplarily generates a serial data stream which can be transported via the multiplex line.

As an alternative to a multiplex line or a data bus, there may be several multiplex lines which in turn are each able to stimulate a part of the stimulation electrodes 122. Exemplarily, the connection lines 125 may comprise four multiplex lines and one ground line. The four multiplex lines each control a part of stimulation electrodes 122 (exemplarily between 50 and 200 or 100) by stimulation impulses (and thus stimulate portions of the retina 103). In this case, there would be four demultiplexers (or control chips) in the second substrate area 120 close to the stimulation electrodes 122 which distribute signals arriving via the individual multiplex lines to the corresponding stimulation electrodes 122.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations, and equivalents as fall within the true spirit and scope of the present invention.

1-32. (canceled)

33. A visual prosthesis for an eye comprising a lens and a retina, comprising:
a first substrate part comprising a coil and being fixable in the lens;
a second substrate part comprising stimulation electrodes and connection lines and being fixable to the retina; and
a third substrate part comprising a processing unit and connecting the first and second substrate parts, the processing unit being electrically connected to the coil and to the stimulation electrodes and being configured to convert a signal generated in the coil by an electromagnetic wave to a stimulation impulse and pass same on via the connection line to the stimulation electrodes,
wherein the first substrate part is foldable relative to the third substrate part and the third substrate part comprises a ring-shaped or crescent-shaped form so that a central area remains open after folding the first substrate part relative to the third substrate part.

34. The visual prosthesis in accordance with claim 33, wherein the first substrate part and the third substrate part are fixed onto each other after folding by means of glue.

35. The visual prosthesis in accordance with claim 33, wherein the first substrate part comprises a film layer structure so that the coil is arranged between two film layers and is hermetically sealed from the environment.

36. The visual prosthesis in accordance with claim 33, wherein a transition region between the first substrate part and the third substrate part comprises a folding supporter and the folding supporter is configured to make folding along a separation line easier.

37. The visual prosthesis in accordance with claim 33, wherein a transition region between the second substrate part and the third substrate part comprises a further folding supporter, wherein the further folding supporter makes folding the third substrate part relative to the second substrate part along a further separation line easier.

38. The visual prosthesis in accordance with claim 33, wherein the first substrate part comprises an annular structure comprising an outer circle edge and an inner circle edge, a first adjusting aid being formed along the inner circle edge,
and wherein the third substrate part comprises a crescent-shaped form comprising an outer crescent circle and an inner crescent circle, a further adjusting aid being formed along the inner crescent circle, wherein the adjusting aid and the further adjusting aid are configured such that, when folding the first relative to the third substrate parts, the adjusting aid and the further adjusting aid are arranged onto each other.

39. The visual prosthesis in accordance with claim 33, comprising metallic structures, wherein the metallic structures allow individual encoding by removing parts of the metallic structures.

40. The visual prosthesis in accordance with claim 39, wherein the metallic structures for encoding are arranged such that the metallic structures remain visible after implantation of the visual prosthesis into the eye.

41. The visual prosthesis in accordance with claim 33, wherein the connection line comprises a multiplex line and the second substrate part comprises a control chip, wherein the control chip is configured to receive signals from the multiplex line and transmit, based on the signals, stimulating impulses to at least part of the stimulation electrodes.

42. A visual prosthesis for an eye comprising a lens and a retina, comprising:
a first substrate part comprising a coil and being fixable in the lens;
a second substrate part comprising stimulation electrodes and connection lines and being fixable to the retina; and
a third substrate part comprising a processing unit and connecting the first and second substrate parts, the processing unit being electrically connected to the coil and the stimulation electrodes and being configured to convert a signal generated in the coil by an electromagnetic wave to stimulation impulses and pass same on to the connection line, wherein the second substrate part comprises a mounting aid which is configured to engage a further adjusting aid connected to the retina.

43. A visual prosthesis for an eye comprising a lens and a retina, comprising:
a first substrate part comprising a coil and being fixable in the lens;
a second substrate part comprising stimulation electrodes and connection lines and being fixable to the retina; and
a third substrate part comprising a processing unit and connecting the first and second substrate parts, the processing unit being electrically connected to the coil and to the stimulation electrodes and being configured to convert a signal generated in the coil by an electromagnetic wave to a stimulation impulse and pass same on via the connection line to the stimulation electrodes, wherein the first substrate part is foldable relative to the third substrate part, and wherein the second substrate part comprises a mounting aid comprising a positioning aid, the mounting aid being configured to engage a further adjusting aid connected to the retina, and the positioning aid being configured to allow relative positioning of a stimulator relative to the further adjusting aid by the positioning aid comprising an inner diameter which is as great as or greater than the adjusting aid.

44. The visual prosthesis in accordance with claim 43, wherein the mounting aid comprises a longitudinal hole and/or a hole comprising a lateral opening so that removing the stimulator is possible without removing the further adjusting aid.

45. The visual prosthesis in accordance with claim 43, wherein the further adjusting aid comprises a retina nail.

46. A method for using a visual prosthesis in accordance with claim 33 or claim 42 or claim 43, comprising:
detecting an image by a camera;
generating a pattern of the detected image;
transmitting the pattern by a transmitter to the visual prosthesis;
generating a stimulation impulse by the processing unit; and
passing the stimulation impulses on to the stimulation electrodes.

47. A method for manufacturing a visual prosthesis for an eye comprising a lens and a retina, comprising:
providing a first substrate part, the first substrate part comprising a coil and being fixable in the lens;
providing a second substrate part comprising stimulation electrodes and connection lines and being fixable to the retina; and
connecting the first substrate part and the second substrate part by a third substrate part, the third substrate part comprising a processing unit so that the coil is connected to the stimulation electrodes, so that a signal generated in the coil by an electromagnetic wave is converted to a stimulation impulse and passed on to the connection lines, wherein the third substrate part may be foldably connected to the first substrate part.

48. A method for attaching a visual prosthesis comprising first, second and third substrate parts at a predetermined position in an eye comprising a lens and a retina, comprising:
folding the first substrate part relative to the third substrate part;
potting the folded first and third substrate parts using silicone;
folding the potted first and third substrate parts along a line which is parallel to a direction of introducing the visual prosthesis into the eye;
introducing the visual prosthesis into the eye;
fixing the folded first and third substrate parts in the lens; and
fixing a second substrate part comprising stimulation electrodes and a connection line to the retina.
